(19) United States
(12) Patent Application Publication
(10) Pub. No.: US 2022/0205996 A1
(43) Pub. Date: Jun. 30, 2022

(54) IMMUNOBLOTTING INSTRUMENT AND CONTROL METHOD FOR CONTROLLING THE SAME

(71) Applicant: Sailejin (Shaoxing) Technology Co., Ltd., Shaoxing (CN)

(72) Inventors: Shipeng Chen, Shaoxing (CN); Liangwei Bao, Shaoxing (CN); Gang Xia, Shaoxing (CN); Jingjing Zhu, Shaoxing (CN); Yinhe Zha, Shaoxing (CN)

(21) Appl. No.: 17/406,114

(22) Filed: Aug. 19, 2021

(30) Foreign Application Priority Data
Dec. 31, 2020 (CN) 202011639404.7

(51) Int. Cl. G01N 33/543; B01L 3/00; B01L 9/00; B01L 7/00; G01N 35/02; G01N 35/00

(52) U.S. Cl. CPC G01N 33/54386; B01L 3/5023; B01L 9/00; B01L 7/52; G01N 35/02; B01L 2200/148; G01N 35/00712; B01L 2300/1838; B01L 2300/1805; B01L 2400/0403; B01L 2400/0622; B01L 3/567

(57) ABSTRACT

An immunoblotting instrument and a control method are provided. The immunoblotting instrument comprises an immunoblotting instrument body, and an immunoblotting instrument control device, an incubation device and a liquid feeding and sucking device that are provided on the immunoblotting instrument body, and further comprises a temperature control device for controlling the incubation environment temperature of the immunoblotting membrane. By setting the temperature control device in the immunoblotting instrument to control the temperature of the reaction environment between the immunoblotting membrane and the reagent, the immunoblotting membrane can contact and react with the reagent under the same and set environmental condition, so that the reliability of the final detection results can be improved; at the same time, each functional module is associated and controlled by the control device, so that the entire immunoblotting process is automatically completed, reducing manual intervention and improving work efficiency.

### CROSS REFERENCE TO RELATED APPLICATIONS

The present application is based on and claims the priority benefits of China application No. 202011639404.7, filed on Dec. 31, 2020. The entirety of the above-mentioned patent application is hereby incorporated by reference herein and made a part of this specification.

### BACKGROUND

Technical Field

The invention relates to the technical field of protein immunoblotting analysis, in particular to an immunoblotting instrument and a control method thereof.

### Description of Related Art

Immunoblotting methods are commonly used to identify certain proteins, can make qualitative and semi-quantitative analysis of protein, and can be extensively used in the fields of life science research and laboratory medicine, such as tumor marker detection and pathogenic microorganism detection. This method transfers the target protein separated by electrophoresis from the gel to a solid support (such as NC membrane or PVDF membrane), and then hybridizes to the specific ligand of the known protein (such as antibody, etc.). Through an enzyme system after color development, the target protein is detected. The immunodetection part successively includes five processes of blocking, primary antibody hybridization, washing, secondary antibody hybridization, and washing, involving multiple steps of liquid addition, liquid aspiration, incubation, and cleaning.

In order to perform the above steps, some immunoblotting instruments can automatically perform the above steps of liquid addition, liquid aspiration, incubation and so on according to a set program. However, by analyzing various types of immunoblotting instruments of the prior art, the inventors found that none of the current immunoblotting instruments can precisely control the incubation reaction conditions of immunoblotting membranes, and the immunoblotting membranes have different effects of the contact reaction with reagents under different environmental conditions, which means that different incubation reaction environments will cause different test results, and ultimately reduce the reliability of the test results.

### SUMMARY

For the problem that the reliability of the detection result of the immunoblotting instrument needs to be further improved in practical application, it is an object of the present application to provide an immunoblotting instrument, which through precise control of immunoblotting membrane incubation and reaction environment condition, can ensure the reliability of detection result. Based on the immunoblotting instrument, another objective of the application is to provide a control method for controlling the immunoblotting instrument. The specific scheme is as follows: an immunoblotting instrument comprises an immunoblotting instrument body, and an immunoblotting instrument control device, an incubation device and a liquid feeding and sucking device that are provided on the immunoblotting instrument body, and further comprises a temperature control device for controlling the incubation environment temperature of the immunoblotting membrane.

Through the above-mentioned technical scheme, the incubation environment temperature in the incubation device can be controlled so as to ensure that the reaction between the immunoblotting membrane and the specific protein in the incubation device can occur under the set environment temperature condition, and the accuracy of the detection result is ensured. Meanwhile, the set temperature can also improve the tightness of binding of the immunoblotting membrane and the specific protein, so that the whole detection efficiency can be improved, and the detection difficulty of the target protein is reduced.

Further, the incubation device comprises a tray rack provided on the immunoblotting instrument body, and a tray detachably mounted on the tray rack for containing the immunoblotting membrane and the reagent; the temperature control device comprises a refrigerator, a refrigeration plate provided with a plurality of refrigeration channels, and a refrigeration conduit communicated with the refrigerator and the refrigeration plate to form a refrigeration circuit, wherein the refrigeration conduit is provided with a liquid pump for driving a cooling liquid in the refrigeration conduit to flow; the refrigerator and the liquid pump are electrically connected to the immunoblotting instrument control device, receive the signal for refrigeration control output by the immunoblotting instrument control device and control the refrigeration power and the flow rate of the cooling liquid; and the refrigeration plate is made of heat conducting metal and is provided at a position on the tray rack supporting the tray.

Through the above technical scheme, the temperature of the tray for storing the immunoblotting membrane can be accurately controlled, so that the incubation reaction temperature of the immunoblotting membrane can be controlled, and the reliability and accuracy of the detection result are ensured.

Further, the liquid feeding and sucking device comprises a holder rack for holding the reagent bottle, a liquid injection conduit for injecting the reagent in the reagent bottle into the tray, a liquid suction conduit for discharging the reagent or waste liquid in the tray, and a peristaltic pump provided on the liquid injection conduit and the liquid suction conduit;
the peristaltic pump is electrically connected to the immunoblotting instrument control device and receives the liquid injection or suction signal output by the immunoblotting instrument control device to act;
the temperature control device further includes an adjustment member for adjusting and controlling the temperature of environment where the holder rack is located.

Through the above technical scheme, the temperature of the reagent injected into the tray can be adjusted, so that the incubation temperature in the tray is always kept at a set value, and the incubation environment condition of the immunoblotting membrane is kept stable.

Further, the holder rack is provided with a plurality of holding cups matched with the reagent bottles in shape and size, the adjustment member comprises a temperature adjusting conduit or temperature adjusting chamber that is provided around the outer side of the holding cups, filled with a heat-conducting medium and communicated with the refrigerator, and a conducting pump is provided between the temperature adjusting conduit or temperature adjusting chamber and the refrigerator;
the conducting pump is electrically connected to the immunoblotting instrument control device, receives the conducting signal of the immunoblotting instrument control device, and controls the flow rate of the heat-conducting medium.

Through the above technical scheme, cold generated by the refrigerator is transferred to the holding cup through the temperature adjusting conduit or temperature adjusting chamber, so that the temperature of reagents in the reagent bottle is maintained within a reasonable range.

Further, the temperature adjusting conduit or temperature adjusting chamber is in communication with the refrigeration conduit to jointly form the refrigeration circuit.

Through the above technical scheme, the temperature of the reagent injected into the tray is consistent with the temperature of the existing reagent in the tray. It is ensured that great mutation of the incubation environment condition in the tray would not happen due to the injection of the reagent.

Further, the temperature control device further comprises a temperature sensor for measuring the temperature of the refrigeration plate that is in signal connection with the control device, and the immunoblotting instrument control device receives a temperature detection signal output by the temperature sensor and adjusts the action of the refrigerator and/or the liquid pump

Through the above technical scheme, the incubation temperature can be monitored and adjusted in real time according to the actual temperature in the tray, and the temperature is ensured to be in a proper range.

Further, the tray rack is movably provided on the immunoblotting instrument body through a mounting seat, and the immunoblotting instrument body is provided with an oscillating assembly for driving the tray rack to reciprocatedly vibrate.

Through the above technical scheme, when the immunoblotting membrane is placed in the tray for incubation, the vibration of the tray rack enables the immunoblotting membrane to be fully contacted with the reagent, so that the reaction efficiency is improved.

Further, the immunoblotting instrument control device comprises a power supply, a control main board and a man-machine interaction assembly. The control main board is provided with a memory and a processor, the memory is loaded with a set program for step execution, and the processor receives and responds to the input of the man-machine interaction assembly to control the actions of the incubation device, the liquid feeding and sucking device and the temperature control device based on the set step execution program in the memory.

Further, the refrigeration conduit comprises a main refrigeration conduit and at least one auxiliary refrigeration conduit that are connected in parallel with each other, two ends of the main refrigeration conduit and of the auxiliary refrigeration conduit are provided with an electrically-controlled multi-way valve electrically connected to the immunoblotting instrument control device; and
the heat conductivity coefficients of the main refrigeration conduit and the auxiliary refrigeration conduit are different from each other.

Through the above technical scheme, after reagent is transmitted through different refrigeration conduits, the temperatures of regent input into the tray can be different, so that different refrigeration conduits can be selected as transmission pipelines of the cooling liquid according to needs, and accurate control of the temperatures in the tray is guaranteed.

Based on the immunoblotting instrument, the invention also provides a control method for controlling the immunoblotting instrument comprising the following steps of:
D1, receiving a start signal;
D2, controlling the operation of a peristaltic pump according to a process set in an immunoassay control table, and injecting the reagent in a reagent bottle into a corresponding tray for reaction;
D3, obtaining a temperature detection signal of each tray and each holding cup;
D4, extracting the temperature thresholds set by the reaction conditions of each tray and each holding cup based on the pre-stored immunoassay control table;
D5, based on the temperature detection signal and the corresponding temperature threshold, outputting a control signal to the corresponding liquid pump in the refrigeration circuit to act; and controlling the liquid pump to stop working when the temperature detection signal reaches the temperature threshold;
D6, controlling the operation of the peristaltic pump according to the process set in the immunoassay control table to extract a reaction solution in the tray into the corresponding reagent bottle to complete the test;
wherein steps D3 to D5 can be executed simultaneously with step D2, or can be set based on the reaction time or specific needs.

Compared with the prior art, the beneficial effects of the present invention are as follows:
By providing the temperature control device in the immunoblotting instrument to control the temperature of the reaction environment of the immunoblotting membrane and the reagent, the immunoblotting membrane can contact and react with the reagent under the same and set environmental condition, so that the reliability of the final detection results can be improved; at the same time, functional modules are associated with one another and controlled by the immunoblotting instrument control device, so that the entire immunoblotting apparatus can automatically complete the detection of the target protein with high efficiency.

### BRIEF DESCRIPTION OF THE DRAWINGS

FIG. 1 is the first overall schematic view of an immunoblotting instrument according to the present invention;

FIG. 2 is the second overall schematic view of an immunoblotting instrument of the present invention;

FIG. 3 is the overall schematic view of an immunoblotting instrument according to the present invention, with a tray omitted;

FIG. 4 is a simplified schematic illustration of the internal structure of the immunoblotting instrument according to the present invention;

FIG. 5 is a schematic diagram of the structural framework of the functional module of the present invention;

FIG. 6 is a schematic diagram of the structure of an incubation device of the present invention;

FIG. 7 is a simplified schematic diagram of the structure of a liquid feeding and sucking device of the present invention;

FIG. 8 is a schematic diagram of a control method of the present invention.

Reference numerals: 1. Immunoblotting instrument Control device; 2. Incubation device; 3. Liquid feeding and sucking device; 4. Temperature control device; 5. Tray rack; 6. Tray; 7. Mounting groove; 8. Holding groove; 9. Reagent bottle; 10. Holder rack; 11.Liquid injection conduit; 12. Liquid suction conduit; 13. Peristaltic pump; 14. Holding cup; 15. Hanging arm; 16. Passing hole; 17. Refrigerator; 18. Refrigeration plate; 19. Refrigeration conduit; 20. Liquid pump; 22. Control main board; 23. Man-machine interaction assembly; 24. Immunoblotting instrument housing; 25. Touch display screen; 26. Temperature adjusting chamber; 27. Heat conducting medium; 28. Conducting pump; 29. Mounting seat; 30. Connecting plate; 31. Drive motor; 32. Via hole; 33. Cam; 34. Roller shaft; 35. Driven gear; 36. Drive gear; 37. Synchronous belt ; 38. Vertical transmission rod.

### DESCRIPTION OF THE EMBODIMENTS

The invention is described in further detail below in conjunction with the examples and drawings, but embodiments of the invention are not limited thereto.

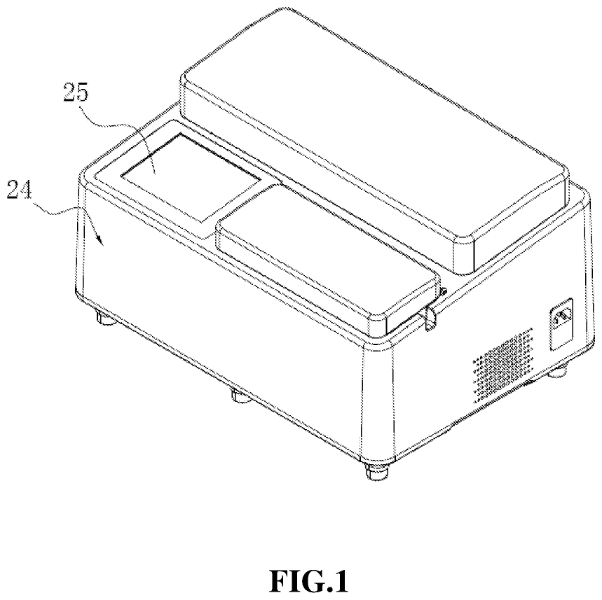

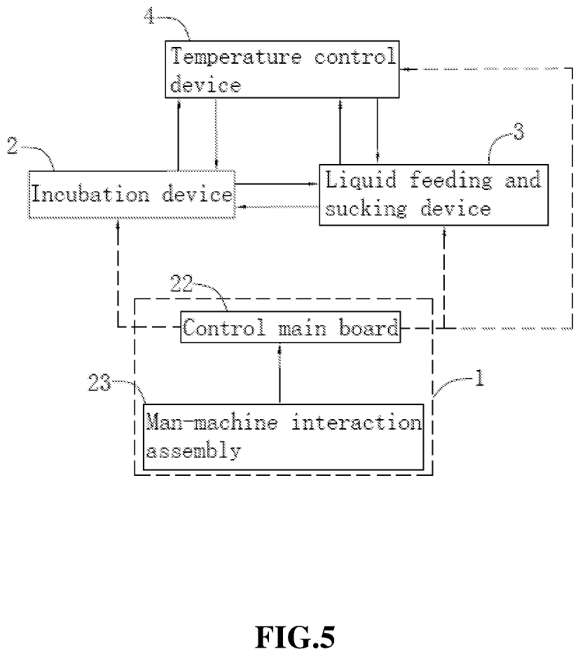

An immunoblotting instrument, as shown in FIGS. 1 and 5, comprises a immunoblotting instrument housing 24, on which there are provided an immunoblotting instrument control device 1, an incubation device 2 and a liquid feeding and sucking device 3. The incubation device 2 is mainly used for providing an appropriate incubation reaction environment for an immunoblotting membrane and a reagent. The liquid feeding and sucking device 3 is mainly used for feeding the reagent, a cleaning liquid into the incubation device 2 or sucking out waste liquid. The immunoblotting instrument control device 1 is used for controlling the actions of the incubation device 2 and the liquid feeding and sucking device 3.

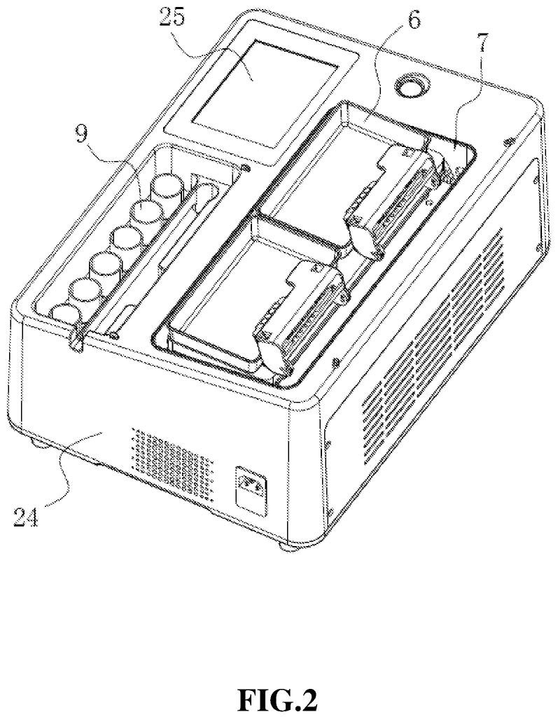

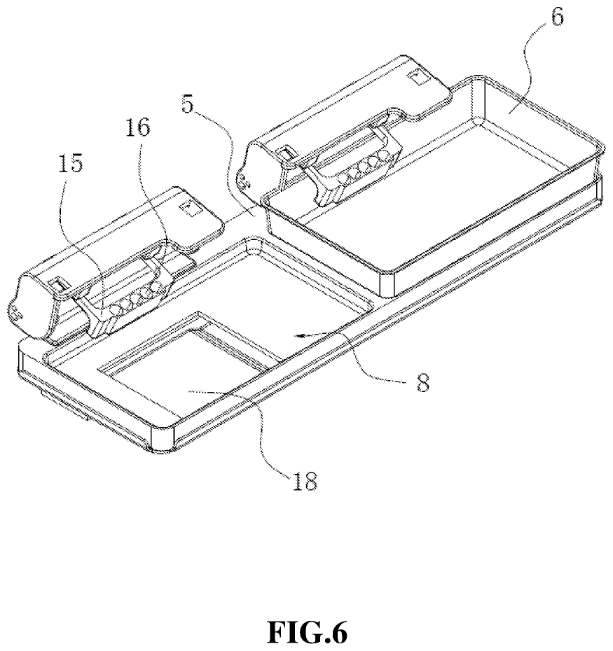

In an embodiment of the present application, the incubation device 2 comprises a tray rack 5 provided on the immunoblotting instrument body, and a tray 6 detachably mounted on the tray rack 5 for storing the immunoblotting membrane and the reagent as shown. As shown in FIGS. 2 and 6, the immunoblotting instrument housing 24 as a whole is cuboid-shaped, and a mounting groove 7 for mounting the tray rack 5 is formed in the top of the immunoblotting instrument housing 24. The mounting groove 7 as a whole is provided in a long plate shape. The shape of the tray rack 5 is matched with that of the mounting groove 7, and the tray rack 5 is mounted in the mounting groove 7. The tray rack 5 is provided with a holding groove 8 for holding the tray 6. There may be a plurality of holding grooves 8 according to needs, and in this embodiment, the number of the holding grooves 8 is two.

In the embodiment, the tray 6 is made of transparent plastics and is convenient to use and detect; and the tray 6 is disposable so as to avoid the cross contamination.

In order to make the reagent injected into the tray 6 sufficiently contact and react with the immunoblotting membrane placed in the tray 6, an oscillating assembly is provided between the tray rack 5 and the top surface of the immunoblotting instrument housing 24.

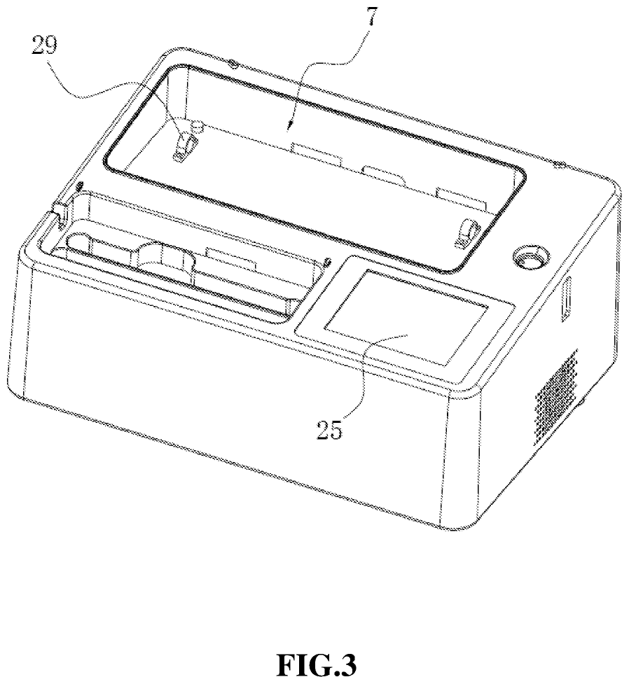

Specifically, in connection with FIG. 3, mounting seats 29 are provided on the top surface of the immunoblotting instrument housing 24 corresponding to two sides of the tray rack 5 in the width direction. A connecting plate 30 is provided at the bottom of the tray rack 5, and is rotatably connected to the mounting seat 29 through a pin shaft, so that the tray rack 5 can rotate by taking a straight line formed by the two mounting seats 29 as a rotating shaft.

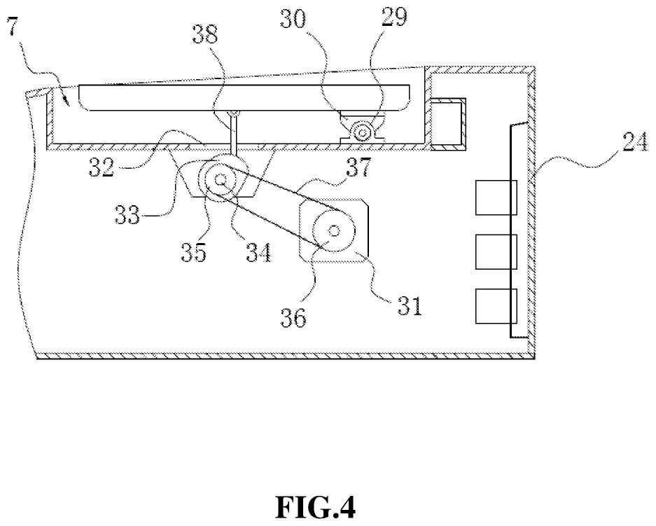

As shown in FIG. 4, the oscillating assembly includes a drive motor 31 provided within the immunoblotting instrument housing 24. The drive motor adopts a servo motor, is fixedly connected to the inner wall of the immunoblotting instrument housing 24 through a mounting hanger, and is electrically connected to the immunoblotting instrument control device 1 through a signal cable and receives the vibration frequency control signal output by the immunoblotting instrument control device to act. A via hole 32 is provided on the top of the immunoblotting instrument housing 24 close to the area of the orthographic projection of tray holder 5 on the top surface of the immunoblotting instrument housing 24. A cam 33 is provided in the via hole 32, and is rotatably connected to the immunoblotting instrument housing 24 through a roller shaft 34 and a bearing sleeved around the peripheral side of the roller shaft 34. The peripheral side of the roller shaft 34 is sleeved with a driven gear 35, and the rotating shaft of the drive motor 31 is sleeved with a driving gear 36. A synchronous belt 37 is wound around the driving gear 36 and the driven gear 35.

A vertical transmission rod 38 is connected between the position close to the edge of the cam 33 and the tray rack 5. The two ends of the vertical transmission rod 38 are respectively hinged with the cam 33 and the tray rack 5. When the cam 33 rotates, the vertical transmission rod drives the tray rack 5 to reciprocatedly vibrate with the mounting seat 29 as a supporting point. In some embodiments, in order to increase the amplitude of the tray rack 5, the connecting plate 30 is provided at the bottom of the pallet rack 5 at a position near its long side.

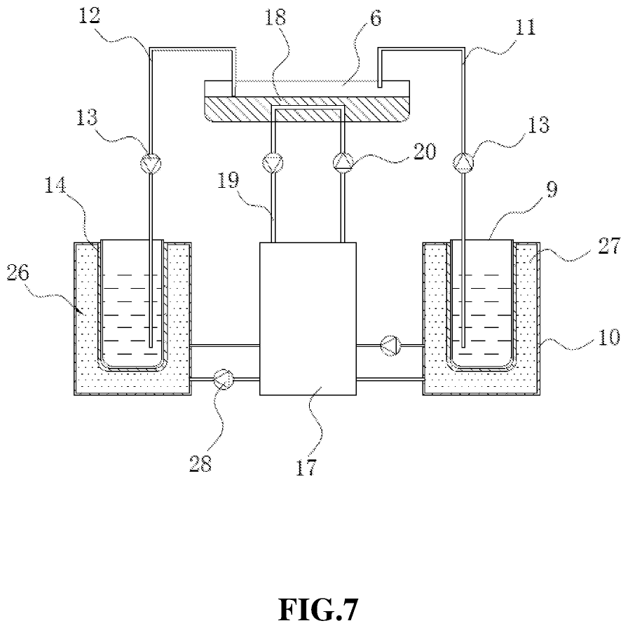

In an embodiment of the present application, in order to inject the reagent in the reagent bottle 9 into the tray 6, the liquid feeding and sucking device 3 comprises a holding rack 10 for holding the reagent bottle 9, a liquid injection conduit 11 for injecting the reagent in the reagent bottle 9 into the tray 6, a liquid suction conduit 12 for discharging the reagent or waste liquid in the tray 6, and a peristaltic pump 13 provided on the liquid injection conduit 11 and the liquid suction conduit 12. In connection with FIGS. 2 and 7, in the embodiment of the present application, the holding rack 10 for the reagent bottle 9 is provided within the immunoblotting instrument body, and a plurality of holding openings are formed at the top of the immunoblotting instrument housing 24. A plurality of holding cups 14 with shapes and sizes matched with those of the reagent bottles 9 are provided on the holding rack 10 for the reagent bottle 9.

In order to smoothly introduce the reagent in the reagent bottle 9 into the tray 6, an opening at an end of the liquid injection conduit 11 far away from the reagent bottle 9 extends to the position above the tray 6. In order to fix the above liquid injection conduit 11, an inverted L-shaped hanging arm 15 is provided on the tray rack 5. An end of the hanging arm 15 is hinged with the tray rack 5, and the other end thereof is a free end and extends to the position right above the tray 6. The hanging arm 15 is locked with the tray rack 5 through a locking piece, such as a clamping block, a bolt and so on. A passing hole 16 for clamping the conduit is provided on the free end of the hanging arm 15, so that the opening of the liquid injection conduit 11 is stably positioned right above the tray 6, and the reagent in the reagent bottle 9 can be directly injected into the tray 6 after being pumped by the peristaltic pump 13.

In an embodiment of the present application, a plurality of reagent bottles 9 are provided, and respectively store a reagent for reaction, a cleaning liquid for cleaning the tray 6, and a waste liquid. Correspondingly, the number of the peristaltic pumps 13 and the number of the liquid injection conduits 11 and the number of the liquid suction conduits 12 are multiple. The above-mentioned liquid injection conduits 11 and the liquid suction conduits 12 each pass through the interior of the immunoblotting instrument housing 24. The peristaltic pumps 13 are all electrically connected to the immunoblotting instrument control device 1, receive and respond to a control signal output from the immunoblotting instrument control device 1 to act. For example, a reagent a in a reagent bottle 9a is injected into a first tray 6 by a liquid injection conduit 11a, a reagent b in a reagent bottle 9b is injected into the first tray 6 by a liquid injection conduit 11b, and waste liquid in the first tray 6 is sucked into a waste liquid collecting bottle or a drain pipe by a liquid suction conduit 12.

In order to maintain the temperature in the tray 6 in a proper range, ensure the activity of protein and ensure that the incubation and reaction can occur efficiently and smoothly, the immunoblotting instrument in the example also comprises a temperature control device 4 for controlling the incubation environment temperature of the immunoblotting membrane.

The temperature control device 4 comprises a refrigerator 17 provided in the immunoblotting instrument housing 24, a refrigeration plate 18 provided with a plurality of refrigeration channels, and a refrigeration conduit 19 communicated with the refrigerator 17 and the refrigeration plate 18 to form a refrigeration circuit. The refrigeration conduit 19 is provided with a liquid pump 20 for driving the cooling liquid in the refrigeration conduit 19 to flow, which may be configured as a peristaltic pump. The refrigerator 17 and the liquid pump 20 are electrically connected to the immunoblotting instrument control device 1, receive the signal for refrigeration control output from the immunoblotting instrument control device 1 to control the refrigeration power of the refrigerator 17 and the pumping rate of the liquid pump 20, so as to control the temperature and the flow rate of the cooling liquid. In an embodiment of the present application, the refrigeration plate 18 is made of a heat-conducting metal, such as aluminum. As shown in FIG. 7, inside the refrigeration plate 18, there are several refrigeration channels for cooling liquid to flow through. The refrigeration channels form liquid inlets and liquid outlets on the refrigeration plate 18. The refrigeration conduit 19 is in communication with the liquid inlets and liquid outlets. In order to transfer the cold of the cooling liquid to the incubation environment of the immunoblotting membrane, the refrigeration plate 18 is provided on the tray rack 5 at a position for supporting the tray 6, that is, at the bottom of the holding groove 8. In a certain embodiment, a heat exchange membrane for improving the cold transfer efficiency is provided on the refrigeration plate 18.

In some embodiments, the refrigeration conduit 19 may be configured as a main refrigeration conduit and at least one auxiliary refrigeration conduit that are connected in parallel with each other. The two ends of the main refrigeration conduit and of the auxiliary refrigeration conduit are provided with an electrically-controlled multi-way valve electrically connected to the immunoblotting instrument control device 1. Based on the above settings, different refrigeration pipelines may be selected to be connected to the refrigeration circuit, and a plurality of the transmission circuit of the cooling liquid may be provided. The heat conductivity coefficients of the main refrigeration conduit and the auxiliary refrigeration conduit are different from each other. For example, the outer side of the main refrigeration conduit may be coated with an insulating layer, such as a heat insulation rubber felt layer. The outer side of the auxiliary refrigeration conduit may be coated with a heat-radiating layer, such as a heat-radiating film; or the auxiliary refrigeration conduit is directly provided as a corrugated pipe. As such, after the reagent is transmitted through different refrigeration conduits 19, the temperature of the cooling liquid that is finally transferred to the tray 6 from different refrigeration conduits 19 will be different, so that different refrigeration conduits 19 (main refrigeration conduit and/or the auxiliary refrigeration duct) can be selected according to the need as the transmission pipeline of the cooling liquid, and the cooling liquid with different temperatures can be adjusted to achieve more diverse and accurate temperatures, so as to realize the precise control of the environment temperature in the tray 6.

Since different reagents need to be injected into the tray 6 at different time according to the preset program or the control signal input through the man-machine interaction assembly during the entire incubation reaction process, in order to avoid sudden changes in the temperature in the tray 6 after the reagents are injected, the temperature control device 4 also includes an adjustment member for adjusting and controlling the environment temperature of the holding rack 10 for the reagent bottle 9.

As shown in FIG. 7, the outer side the holding rack 10 for holding the reagent bottle 9 is provided with a sealing housing. A sealed temperature adjusting chamber 26 is formed between the sealed housing and the outer wall of the holding cup 14, and is filled with heat conducting medium 27, such as clear water. The temperature adjusting chamber 26 is communicated with the refrigerator 17, and a conducting pump is provided between the temperature adjusting chamber 26 and the refrigerator 17. The conducting pump is electrically connected to the immunoblotting instrument control device 1, receives a conducting signal of the immunoblotting instrument control device 1 to control the heat conducting medium to flow into the temperature adjusting chamber 26 and control the flow rate of the heat conducting medium 27. In this embodiment, the holding cup 14 is made of heat conducting material, such as stainless steel. The cold produced by the refrigerator 17 is transferred to the temperature adjusting chamber 26 via the heat conducting medium 27 to adjust the temperature in the temperature adjusting chamber 26 and the holding cup 14.

By adopting the technical scheme, the temperature of the reagent in the reagent bottle 9 placed in the holding cup 14 can be maintained within a set range, so that the temperature in the tray 6 cannot change suddenly when the reagent is injected into the tray 6.

In another embodiment, refrigeration of the holding cup 14 may be achieved by winding a temperature adjusting conduit communicating with the refrigerator 17 around the outer side of the holding cup 14.

In some embodiments, the refrigeration conduit 19, the refrigerator 17, the liquid pump 20 and the temperature adjusting chamber 26 in the present application jointly form a refrigeration circuit, that is, the temperature adjustment chamber 26 and the refrigeration plate 18 are connected through the refrigeration conduit 19 and the liquid pump 20. In this way, the temperature of the reagent injected into the tray 6 is consistent with that of the existing reagent in the tray 6, and the incubation environment condition in the tray 6 is ensured not to be greatly changed due to the injection of the reagent.

To accurately control the temperature of the reagent in the tray 6, the temperature control device 4 in the present application further comprises a temperature sensor for measuring the temperature of the refrigeration plate 18. The temperature sensor is electrically connected to the immunoblotting instrument control device 1, collects the temperature on the refrigeration plate 18 and outputs the temperature detection signal to the immunoblotting instrument control device 1. Based on the temperature detection signal, the immunoblotting instrument control device 1 adjusts the action of the refrigerator 17 and/or the liquid pump 20. For example, when the temperature in the tray 6 is too high, the refrigeration power of the refrigerator 17 or the pumping power of the liquid pump 20 is increased, so that the temperature of the cooling liquid is reduced or the flow rate of the cooling liquid is accelerated, the heat in the tray 6 is taken away more quickly to realize the accurate control of the temperature, and the activity of the target protein in the tray 6 is ensured.

As shown in FIG. 5, in this application, the immunoblotting instrument control device 1 comprises a power supply, a control main board 22 and a man-machine interaction assembly 23. The control main board 22 is provided with a memory and a processor. The memory is loaded with a set program for step execution, and different detection programs are stored for different proteins to be detected. The processor is in connection with the signal output end of the man-machine interaction assembly 23, receives and responds to the operation instruction input by the man-machine interaction assembly 23, and retrieves the set step execution program from the memory, and control the actions of the incubation device 2, the liquid feeding and sucking device 3 and the temperature control device 4. The processor may adopt ARM, FPGA or SCM. The man-machine interaction assembly includes a touch display screen 25 which can receive input instructions from an operator and can also display the operating status of each working component in the immunoblotting instrument. As shown in FIG. 1, an upper side of the immunoblotting instrument is provided with a chamfered angle, and the touch screen 25 is arranged at the chamfered angle to facilitate the operator to view relevant information and input instructions.

It should be noted that the plurality of liquid feeding and sucking devices 3 and temperature control devices 4 are independently provided to provide different incubation and reaction temperatures to the plurality of trays 6.

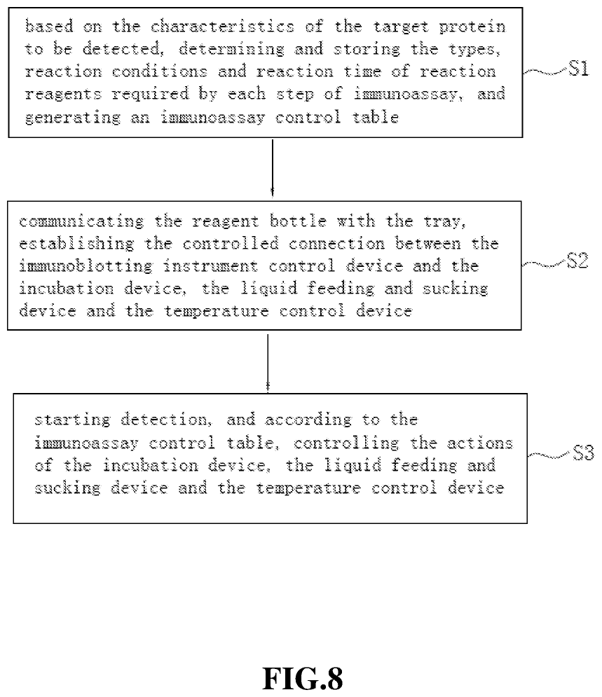

Based on the immunoblotting instrument, the invention also provides a control method for controlling the immunoblotting instrument, as shown in FIG. 8, which comprises the following steps:
S100, based on the characteristics of the target protein to be detected, determining and storing the types, reaction conditions and reaction time of reaction reagents required by each step of immunoassay, and generating an immunoassay control table;
S200, communicating the reagent bottle 9 with the tray 6, establishing the electrical connection between the immunoblotting instrument control device 1 and the incubation device 2, the liquid feeding and sucking device 3 and the temperature control device 4;
S300, starting detection, and according to the immunoassay control table, controlling the actions of the incubation device 2, the liquid feeding and sucking device 3 and the temperature control device 4.

In the above step S100, based on the characteristics of various target proteins to be detected, a corresponding detection program is stored in advance in the memory of the immunoblotting instrument control device 1, such as the temperature in the tray 6, the time for filling reagent, the time for reaction and the time for feeding the liquid, and then according to the above-mentioned steps a correspondent immunoassay control table is generated. The above-mentioned immunoassay control table is actually a control code that can be directly stored in the single-chip microcomputer or a memory chip connected with it.

In the step S200, the operator communicates different liquid injection conduits 11 and liquid suction conduits 12 with corresponding reagent bottles 9 before detection, and then detects whether the instrument can be controlled normally or not. When the detection is completed, the instrument is started to begin detection, The immunoblotting instrument control device 1 in the instrument can control the operation of the peristaltic pump 13 according to a set control program so as to regularly and quantitatively extract and inject the reagent into the tray 6, and suck the reagent or waste liquid out of the tray 6 at a set time; meanwhile, it can stabilize the reaction temperature of the whole process, so that the normal reaction is ensured; manual intervention is not needed in the detection process, so that the detection can be efficiently and automatically finished with a high efficiency. Since the reaction environment is consistent, the reliability of the detection result is improved.

Based on the above-mentioned steps, as detailed, the above-mentioned control method for controlling an immunoblotting instrument further includes the following steps:
D1, receiving a start signal that may be collected and input from the man-computer interaction assembly 23;
D2, controlling the operation of a peristaltic pump 13 according to a process set in an immunoassay control table, and injecting the reagent in a reagent bottle 9 into a corresponding tray 6 for reaction;
D3, obtaining a temperature detection signal of each tray 6 and each holding cup 14;
D4, extracting the temperature thresholds set by the reaction conditions of each tray 6 and each holding cup 14 based on the pre-stored immunoassay control table;
D5, based on the temperature detection signal and the corresponding temperature threshold, outputting a control signal to the corresponding liquid pump 20 in the refrigeration circuit to act; and controlling the liquid pump 20 to stop working when the temperature detection signal reaches the temperature threshold;
D6, controlling the operation of the peristaltic pump 13 according to the process set in the immunoassay control table to extract a reaction solution in the tray 6 into the corresponding reagent bottle 9 to complete the test;
wherein steps D3 to D5 can be executed simultaneously with step D2, or can be set based on the reaction time or specific needs.

The foregoing description is merely preferred embodiments of the invention, The scope of the invention is not limited to the embodiments described above. It should be noted that modifications and amendments, which fall within the scope of the present invention, will also be considered to those of ordinary skill in the art without departing from the principles of the present invention as falling within the scope of the invention.

What is claimed is:

1. An immunoblotting instrument, characterized by comprising an immunoblotting instrument body, and on the immunoblotting instrument body there are provided with:
an immunoblotting instrument control device (1) for controlling the operation of various devices of the immunoblotting instrument;
an incubation device (2) provided on the immunoblotting instrument body for holding an immunoblotting membrane and a reagent;
a liquid feeding and sucking device (3) for feeding the reagent to the incubation device (2) or sucking out the reagent in the incubation device (2); and
a temperature control device (4) electrically connected to the immunoblotting instrument control device (1), for controlling an incubation environment temperature of the immunoblotting membrane in the incubation device (2).

2. The immunoblotting instrument of claim 1, wherein the incubation device (2) comprises a tray rack (5) provided on the immunoblotting instrument body, and a tray (6) detachably mounted on the tray rack (5) for containing the immunoblotting membrane and the reagent;
the temperature control device (4) comprises a refrigerator (17), a refrigeration plate (18) provided with a plurality of refrigeration channels, and a refrigeration conduit (19) communicated with the refrigerator (17) and the refrigeration plate (18) to form a refrigeration circuit, wherein the refrigeration conduit (19) is provided with a liquid pump (20) for driving a cooling liquid in the refrigeration conduit (19) to flow;
the refrigerator (17) and the liquid pump (20) are electrically connected to the immunoblotting instrument control device (1), receive a signal for refrigeration control output by the immunoblotting instrument control device (1), and control a refrigeration power and a flow rate of the cooling liquid; and
the refrigeration plate (18) is made of heat conducting metal and is provided at a position on the tray rack (5) supporting the tray (6).

3. The immunoblotting instrument of claim 2, wherein the liquid feeding and sucking device (3) comprises a holder rack (10) provided on the immunoblotting instrument body for holding a reagent bottle (9), a liquid injection conduit (11) for injecting the reagent in the reagent bottle (9) into the tray (6), a liquid suction conduit (12) for discharging the reagent or a waste liquid in the tray (6), and a peristaltic pump (13) provided on the liquid injection conduit (11) and the liquid suction conduit (12);
the peristaltic pump (13) is electrically connected to the immunoblotting instrument control device (1), and receives a liquid injection or suction signal output by the immunoblotting instrument control device (1) to act;
the temperature control device (4) further comprises an adjustment member for adjusting and controlling a temperature of environment where the holder rack (10) is located.

4. The immunoblotting instrument of claim 3, wherein the holder rack (10) is provided with a plurality of holding cups (14) matched with the reagent bottles (9) in shape and size, the adjustment member comprises a temperature adjusting conduit or temperature adjusting chamber (26) that is provided around an outer side of the holding cups (14), filled with a heat-conducting medium (27) and communicated with the refrigerator (17), and a conducting pump (28) is provided between the temperature adjusting conduit or temperature adjusting chamber (26) and the refrigerator (17);
the conducting pump is electrically connected to the immunoblotting instrument control device (1), receives a conducting signal of the immunoblotting instrument control device (1), and controls a flow rate of the heat-conducting medium (27).

5. The immunoblotting instrument of claim 4, wherein the temperature adjusting conduit or temperature adjusting chamber (26) is in communication with the refrigeration conduit (19) to jointly form the refrigeration circuit.

6. The immunoblotting instrument of claim 2, wherein the temperature control device (4) further comprises a temperature sensor for measuring the temperature of the refrigeration plate (18) that is electrically connected to the immunoblotting instrument control device (1), and the immunoblotting instrument control device (1) receives a temperature detection signal output by the temperature sensor and adjusts the action of the refrigerator (17) and/or the liquid pump (20).

7. The immunoblotting instrument of claim 2, wherein the tray rack (5) is movably provided on the immunoblotting instrument body through a mounting seat, and the immunoblotting instrument body is provided with an oscillating assembly for driving the tray rack (5) to reciprocate around a set axial direction.

8. The immunoblotting instrument of claim 1, wherein the immunoblotting instrument control device (1) comprises a power supply, a control main board (22) and a man-machine interaction assembly (23), the control main board (22) is provided with a memory and a processor, the memory is loaded with a set program for step execution, and the processor receives and responds to an input of the man-machine interaction assembly (23) to control the actions of the incubation device (2), the liquid feeding and sucking device (3) and the temperature control device (4) based on the set step execution program in the memory.

9. The immunoblotting instrument of claim 2, wherein the refrigeration conduit (19) comprises a main refrigeration conduit and at least one auxiliary refrigeration conduit that are connected in parallel with each other, and two ends of the main refrigeration conduit and of the auxiliary refrigeration conduit are provided with an electrically-controlled multi-way valve electrically connected to the immunoblotting instrument control device (1);
the heat conductivity coefficients of the main refrigeration conduit and the auxiliary refrigeration conduit are different from each other.

10. A control method of an immunoblotting instrument characterized by comprising the following steps of:
D1, receiving a start signal;
D2, controlling the operation of a peristaltic pump (13) according to a process set in an immunoassay control table, and injecting the reagent in a reagent bottle (9) into a corresponding tray (6) for reaction;
D3, obtaining a temperature detection signal of each tray (6) and each holding cup (14);
D4, extracting temperature thresholds set by the reaction conditions of each tray (6) and each holding cup (14) based on the pre-stored immunoassay control table;
D5, based on the temperature detection signal and the corresponding temperature threshold, outputting a control signal to a corresponding liquid pump (20) in the refrigeration circuit to act; and controlling the liquid pump (20) to stop working when the temperature detection signal reaches the temperature threshold;
D6, controlling operation of the peristaltic pump (13) according to the process set in the immunoassay control table to extract a reaction solution in the tray (6) into the corresponding reagent bottle (9) to complete a test;
wherein steps D3 to D5 are able to be executed simultaneously with step D2, or able to be set based on a reaction time or specific needs.
